(19) United States
(12) Patent Application Publication
(10) Pub. No.: US 2007/0156162 A1
(43) Pub. Date: Jul. 5, 2007

(54) Combination tissue dissector and surgical implant inserter

(76) Inventor: Gene W. Kammerer, East Brunswick, NJ (US)

Correspondence Address: BODNER & O'ROURKE, LLP, 425 BROADHOLLOW ROAD, SUITE 108, MELVILLE, NY, 11747, US

(21) Appl. No.: 11/322,142

(22) Filed: Dec. 29, 2005

(51) Int. Cl. A61B 17/32

(52) U.S. Cl. 606/174

(57) ABSTRACT

A surgical instrument for dissecting tissue and inserting a surgical implant device thereat includes first and second blades which are pivotally joined together. Each of the first and second blades has an elongated length which includes a first section and a second section, the first section of each blade including a free end having a flattened tip for blunt dissection of tissue and a non-cutting, clamping portion situated adjacent to and encompassing the flattened tip for selectively grasping a surgical implant device. The second section of each blade includes a finger ring for grasping and manipulation by a surgeon. The blades are pivotally joined together at the junctures of the first and second sections to allow the first and second blades to pivotally move with respect to one another within a plane of pivotal movement. The non-cutting, clamping portions of each blade has a clamping face, and the clamping faces of the blades reside in parallel planes and are angled to the plane of pivotal movement of the blades.

### BACKGROUND OF THE INVENTION

1. Field of the Invention

The present invention generally relates to surgical instruments, and more specifically relates to surgical instruments used in dissecting tissue and inserting a surgical implant at the dissection site.

2. Description of the Prior Art

In a surgical procedure in which an implant, such as a surgical mesh, is placed to support or reinforce tissue, such as in pelvic organ prolapse repair using a vaginal stabilizer in the form of an elongated mesh, there are two critical steps. The first is the dissection of the tissues, and the second is the laying down or placement of the mesh. Surgeons use a variety of surgical instruments to accomplish these tasks. The choice of instruments is dependent on the patient's anatomy into which the mesh will be implanted and the type of dissection which will be performed. In many instances, the dissection performed is “blunt” dissection. Blunt dissection is where no sharp cutting of the tissue is performed. An instrument is pushed between two layers of tissue, and the advancement of the instrument separates the tissue layers generally along the lines of a weak connection or bond between the two layers. In some cases, additional separation is required to make the area wider, and this can be accomplished by using a scissor. The scissor is closed, inserted between the tissue layers, and then opened while between the layers. In this procedure, there is no shearing or cutting of the tissue.

After the tissue has been dissected to form a pocket or channel, the mesh is then inserted therein. Different surgical instruments are used to insert the mesh. Sometimes a surgeon will use tweezers, and sometimes a hemostat, or grasper. The instrument used to insert the mesh may be curved or straight. The tweezers often have serrations at their tip, or a pin to hold the mesh securely during the implantation procedure. The hemostat also has serrations to grasp the mesh.

There are many disadvantages in using the above-described surgical instruments. For example, there are many sizes to the tweezers and scissors, and the surgeon must choose the correct instrument that is suitable for use with the particular procedure being performed. For example, if the instrument is oversized, the pocket or channel can be made too large, causing the implant to possibly move prior to incorporation by the in growing tissue. Also, the curvatures of the surgical devices are not always optimal for the attempted dissection. Tweezers are almost always straight and during insertion cannot follow the contours of some anatomy. Hemostats generally have 90° angles near the distal end. This can lead to unwanted enlargements of a narrow channel in the tissue or difficult placement of the implant and removal of the instrument.

Additionally, the means for grasping the mesh situated at the instrument tip sometimes snag the mesh and damage it, or fail to release it completely. The mesh then is pulled out of the dissected channel when the instrument is retrieved. Conversely, the instrument should be capable of holding the mesh firmly while it is being inserted and maneuvered, and then releasing it cleanly without enlarging the pocket or channel.

Furthermore, these surgical instruments do not have any measurement indications on them and, therefore, a secondary device, such as ruler, has to be used to identify the size and proportions of the anatomical area. This second instrument is placed or pushed into the channel to determine the length, depth and width of the dissected pocket or channel. The measurement is then transferred to the mesh for cutting and proper sizing. Because the rulers that are available are wide, this can inadvertently increase the size of the pocket or channel by additional blunt dissection when the ruler is inserted into the dissected channel. When this occurs, the mesh will not fit correctly and may not stay as placed until tissue in-growth in the mesh has occurred. Misalignment of the mesh at the surgical site can result in a procedural failure. Also, if the channel is sized incorrectly to properly seat the mesh, the mesh could become folded. A folded mesh could cause a lump or hard spot that may lead to patient discomfort later. As an alternative to inserting a ruler into the pocket or channel, a narrow surgical instrument can be used to record the channel length or depth. The distance recorded on the instrument must then be measured by a ruler and transferred to the implant. Making measurements in this manner requires numerous steps which increases surgical time and provides the chance for error.

### OBJECTS AND SUMMARY OF THE INVENTION

It is an object of the present invention to provide a surgical instrument which performs multiple surgical procedural tasks.

It is another object of the present invention to provide a surgical instrument that can perform multiple surgical procedural tasks which include the ability to do a variety of blunt dissection techniques.

It is still another object of the present invention to provide a surgical instrument which can instantly grasp and then instantly release a material along its full holding length.

It is yet another object of the present invention to provide a surgical instrument which can release a grasped material with minimal movement of its grasping portion.

It is a further object of the present invention to provide a surgical instrument which can hold an implantable material with a significant force and can open its grasping portion against a significant force.

It is yet a further object of the present invention to provide a surgical instrument which has an atraumatic grasping structure that can hold an implantable material firmly.

It is still a further object of the present invention to provide a surgical instrument which can be used to measure distances or lengths of dissected tissue pockets or channels or other anatomical structures.

It is still another object of the present invention to provide a surgical instrument which may be used as a scissor to cut tissue or other material.

In accordance with one form of the present invention, a surgical instrument for dissecting tissue and inserting a surgical implant device thereat includes a first blade having an elongated length including a first section and a second section disposed opposite the first section. The first section of the first blade includes a free end having preferably a flattened tip for blunt dissection of tissue, and a non-cutting, clamping portion situated adjacent to and encompassing the flattened tip for selectively grasping a surgical implant device. The second section includes a finger ring for grasping and manipulation by a surgeon.

The second blade also has an elongated length including a first section and a second section disposed opposite the first section. The first section of the second blade includes a free end also preferably having a flattened tip for blunt dissection of tissue, and a non-cutting, clamping portion situated adjacent to and encompassing the flattened tip for selectively grasping a surgical implant device. The second section of the second blade also includes a finger ring for grasping and manipulation by a surgeon.

The first blade and the second blade are pivotally joined together at the junctures of the first and second sections of each blade to allow the first and second blades to pivotally move with respect to one another within a plane of pivotal movement. The non-cutting, clamping portion of each of the first and second blades has a clamping face. The clamping face of the first blade and the clamping face of the second blade are arranged to face each other and to selectively engage and hold therebetween a surgical implant device. The clamping faces of the first and second blades reside in parallel planes and are angled to the plane of pivotal movement of the blades.

The first and second blades are relatively pivotable between at least a first position, wherein the clamping faces of the first and second blades are spaced from each other a first predetermined distance to grasp and hold securely therebetween a surgical implant device, and a second position, wherein the clamping faces of the first and second blades are spaced from each other a second predetermined distance to release a surgical implant device situated therebetween, where the first predetermined distance is less than the second predetermined distance.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIG. 1 is a top perspective view of one form of a combination tissue dissector and surgical implant inserter formed in accordance with the present invention.

FIG. 2 is a side view of the combination tissue dissector and surgical implant inserter shown in FIG. 1, shown holding a surgical implant device.

FIG. 3 is a front view of the combination tissue dissector and surgical implant inserter of the present invention shown in FIGS. 1 and 2.

FIG. 4 is a front view of a portion of the combination tissue dissector and surgical implant inserter shown in FIGS. 1-3.

FIG. 5 is a side view of a portion of the combination tissue dissector and surgical implant inserter shown in FIG. 1-4, shown holding a surgical implant device.

FIG. 6 is a top perspective view of a second embodiment of the combination tissue dissector and surgical implant inserter formed in accordance with the present invention.

FIG. 7 is a front view of a portion of the combination tissue dissector and surgical implant inserter of the present invention shown in FIG. 6.

FIG. 8 is a transverse cross-sectional view of a combination tissue dissector and surgical implant inserter shown in FIGS. 6 and 7, and shown holding a surgical implant device.

FIG. 9 is a side view of a portion of the combination tissue dissector and surgical implant inserter of the present invention shown in FIGS. 6-8.

FIG. 10 is a front view of a portion of the combination tissue dissector and surgical implant inserter of the present invention illustrating structure for holding a surgical implant device in place.

FIG. 11 is a side view of a portion of the combination tissue dissector and surgical implant inserter shown in FIG. 10.

FIG. 12 is a front view of a portion of the combination tissue dissector and surgical implant inserter of the present invention illustrating an alternative form of structure for holding a surgical implant device in place.

FIG. 13 is an opposite rear view of a portion of the combination tissue dissector and surgical implant inserter shown in FIG. 12.

FIG. 14 is a front view of a portion of a combination tissue dissector and surgical implant inserter of the present invention, illustrating another form of structure for holding a surgical implant device in place.

FIG. 15 is an opposite rear view of a portion of the combination tissue dissector and surgical implant inserter of the present invention shown in FIG. 14.

FIG. 16 is a front perspective view of a third embodiment of the combination tissue dissector and surgical implant inserter formed in accordance with the present invention.

FIG. 17 is an enlarged front perspective view of a portion of the combination tissue dissector and surgical implant inserter of the present invention shown in FIG. 16.

### DETAILED DESCRIPTION OF THE PREFERRED EMBODIMENTS

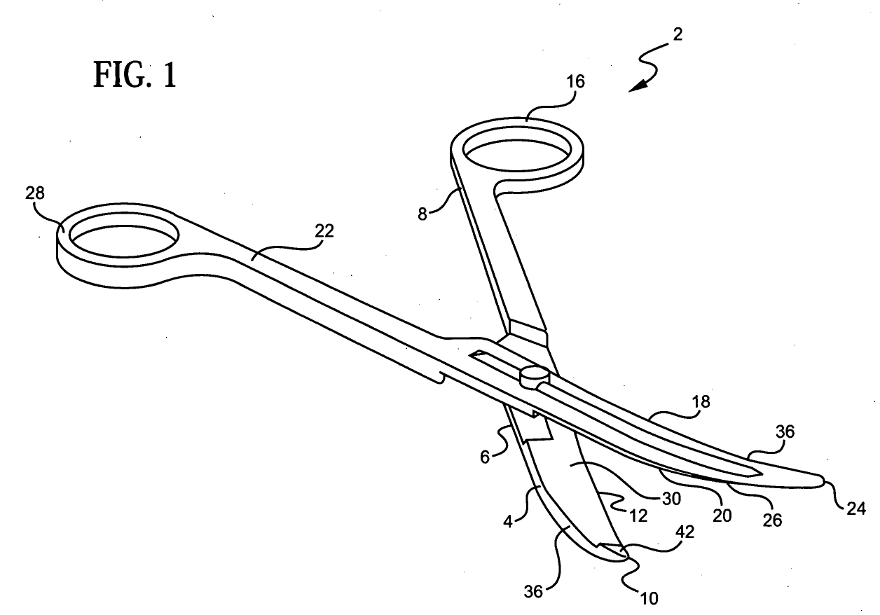

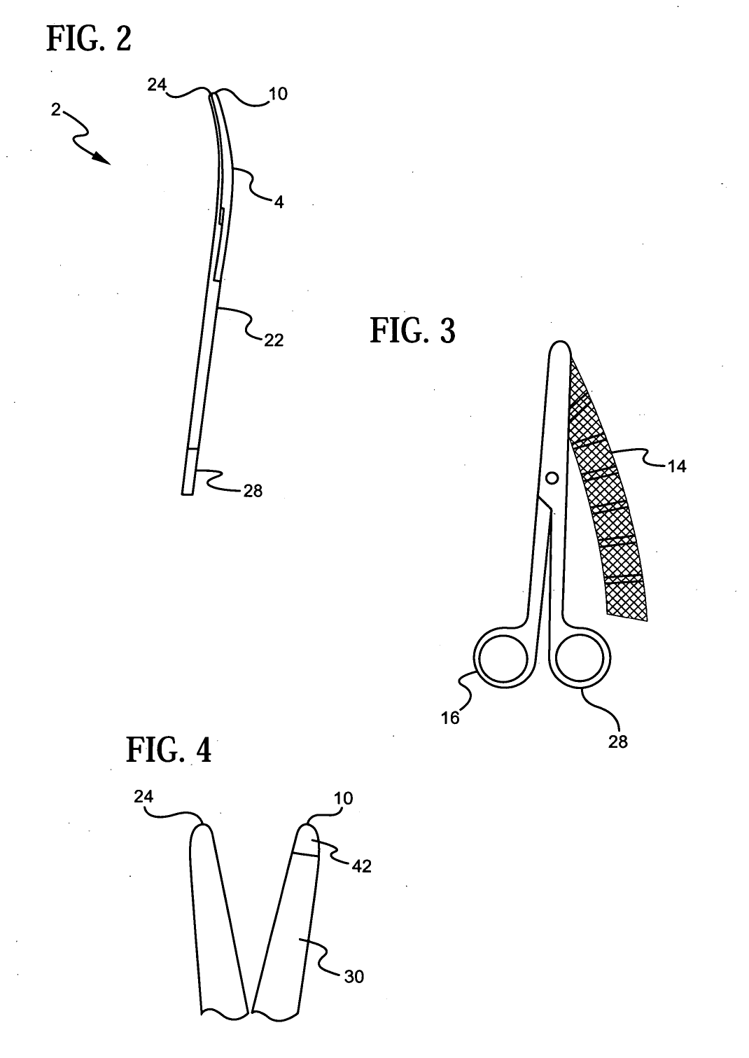

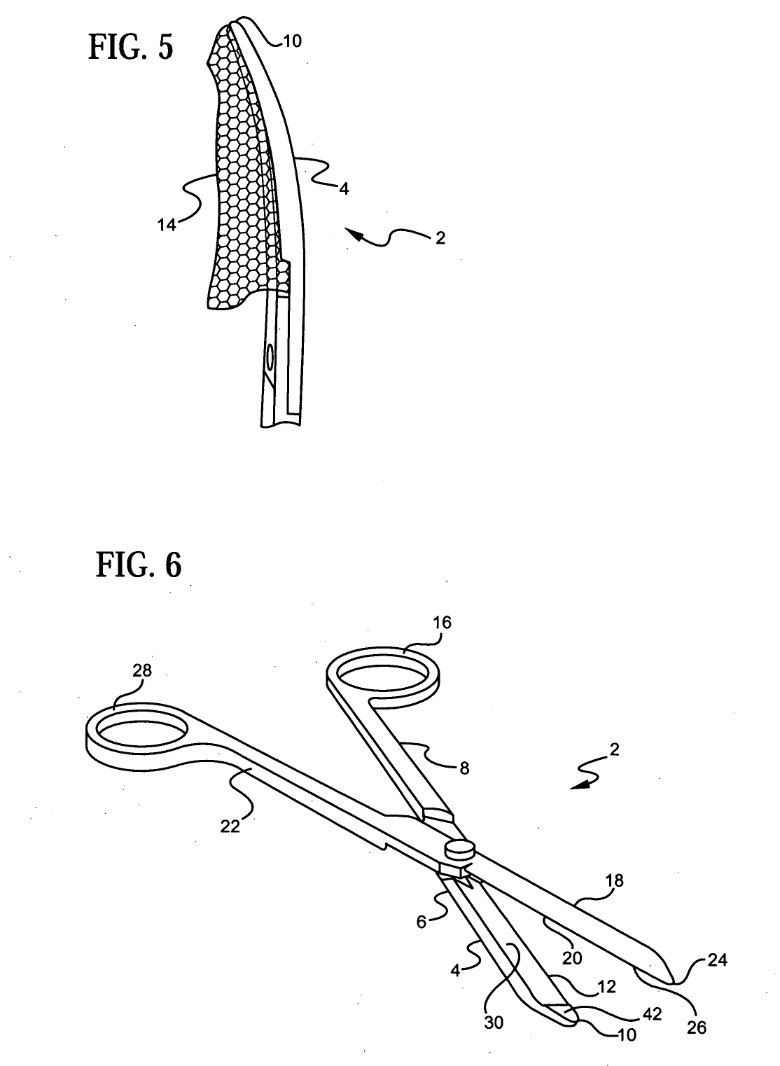

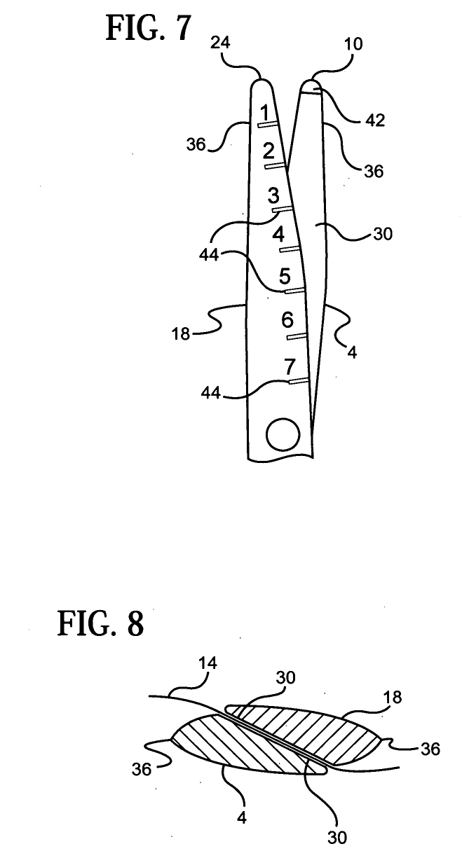

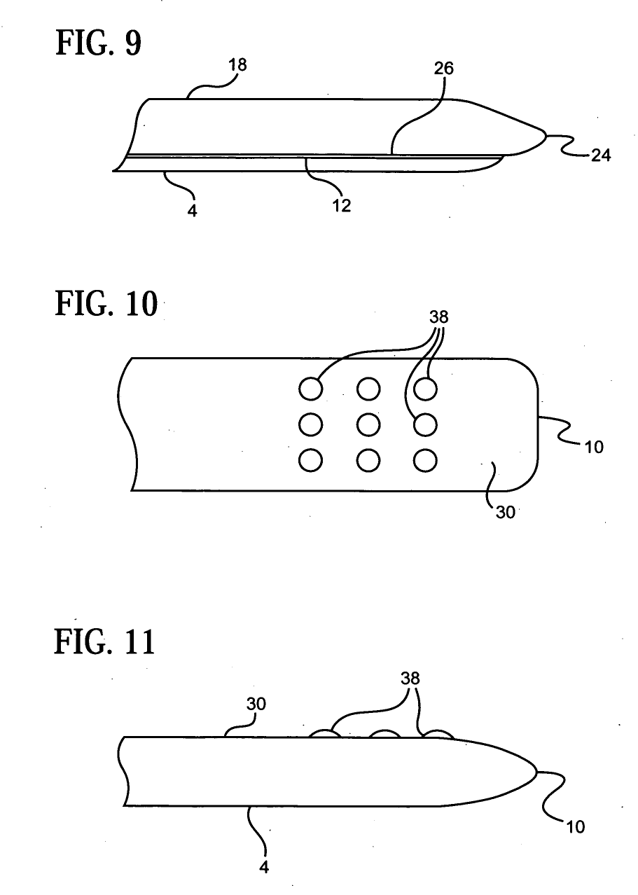

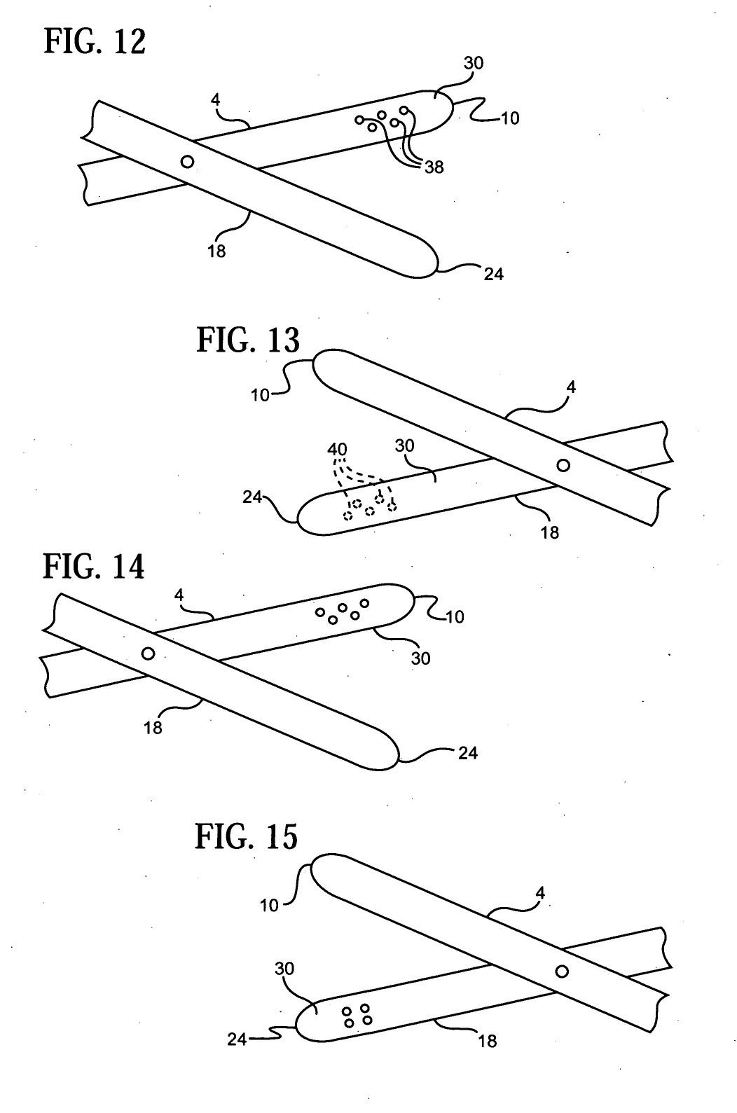

Referring to FIGS. 1-17, it will be seen that a surgical instrument 2 for dissecting tissue and inserting a surgical implant device at the dissection site has the general shape of a scissor or forceps. More specifically, the surgical instrument 2 includes a first blade 4 having an elongated length including a first section 6 and a second section 8 disposed opposite the first section 6. The first section 6 of the first blade 4 includes a free end having a preferably flattened tip 10 for blunt dissection of tissue, and a non-cutting, clamping portion 12 situated adjacent to and encompassing the flattened tip 10. The non-cutting, clamping portion 12 is provided for selectively grasping a surgical implant device, such as a surgical mesh 14. The second section 8 of the first blade 4 includes a finger ring 16 for grasping and manipulation by a surgeon.

The surgical instrument also includes a second blade 18 which, similarly, has an elongated length including a first section 20 and a second section 22 disposed opposite the first section 20. The first section 20 of the second blade 18 also similarly includes a free end having a preferably flattened tip 24 for blunt dissection of tissue, and a non-cutting, clamping portion 26 situated adjacent to and encompassing the flattened tip 24, also for selectively grasping a surgical implant device. The second section 22 of the second blade 18, like the first blade 4, includes a finger ring 28 for grasping and manipulation by a surgeon. Thus, the second sections 8, 22 of the surgical instrument 2, together, with their finger rings 16, 28 define a handle for grasping the instrument.

The first blade 4 and the second blade 18 are pivotally joined together at the junctures of the first and second sections 6, 8, 20, 22 of each blade to allow the first and second blades to pivotally move with respect to one another within a plane of pivotal movement. The non-cutting, clamping portion 12, 26 of each of the first and second blades has a clamping face 30. The clamping face 30 of the first blade 4 and the clamping face 30 of the second blade 18 are arranged to face each other and to selectively engage and hold therebetween a surgical implant device. The clamping faces 30 of the first and second blades 4, 18 reside in parallel planes and are angled to the plane of pivotal movement of the blades.

The first and second blades 4, 18 are relatively pivotable between at least a first position, wherein the clamping faces 30 of the first and second blades are spaced from each other a first predetermined distance to grasp and hold securely therebetween a surgical implant device 14, and a second position, wherein the clamping faces 30 of the first and second blades are spaced from each other a second predetermined distance which is greater than the first predetermined distance to release a surgical implant device 14 situated therebetween.

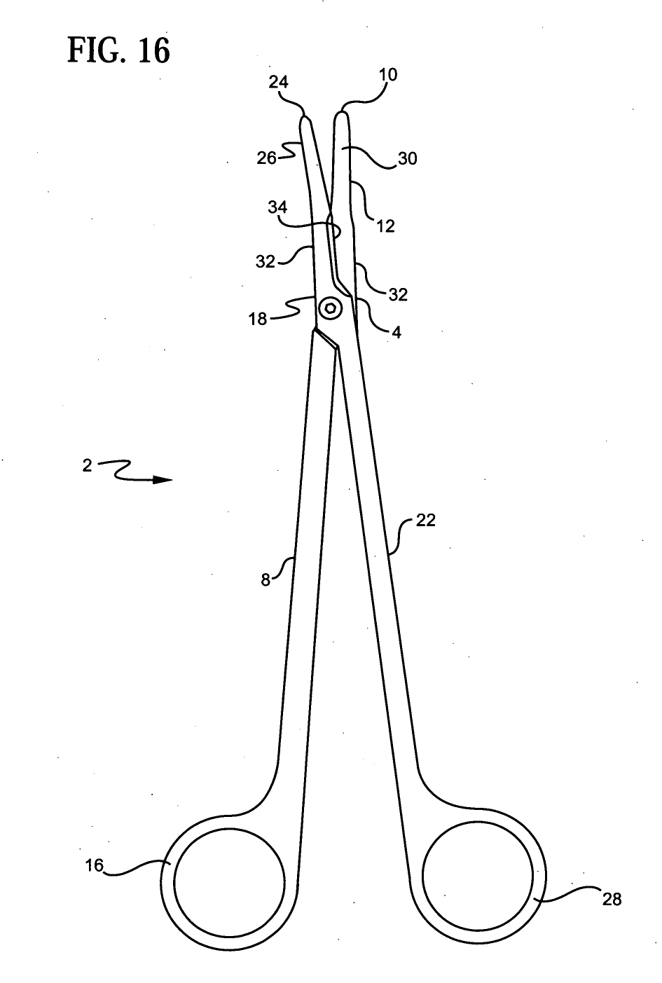

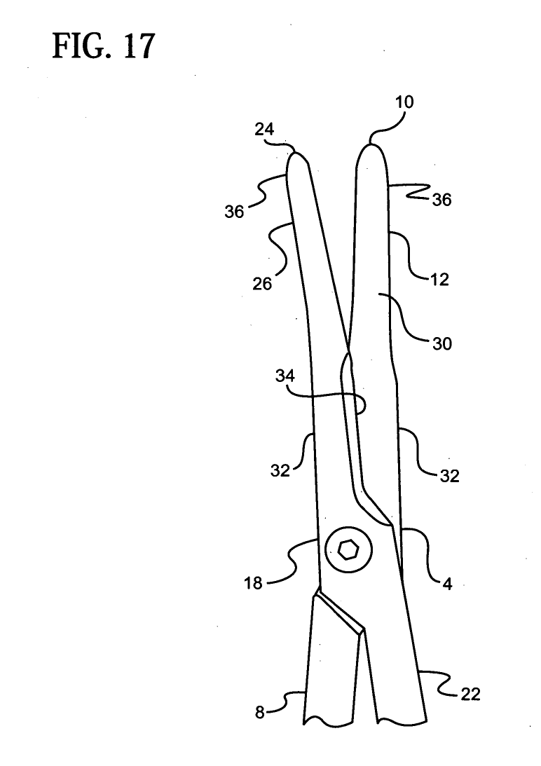

Preferably, the planes in which each of the clamping faces 30 of the first and second blades 4, 18 respectively reside are disposed at preferably between about 20 degrees and about 70 degrees, and more preferably, at about a 40 degree angle, to the plane of pivotal movement of the first and second blades. Furthermore, the non-cutting, clamping portions 12, 26 of the first and second blades 4, 18 may be curved, as shown in FIGS. 1-5, as some surgeons prefer using a surgical instrument having a curved blade as opposed to a straight blade, as shown in FIGS. 6-8 and in FIGS. 16 and 17 of an alternative straight bladed embodiment of the present invention. With a curved blade, the plane of pivotal movement would be similarly curved.

The first section 6, 20 of each of the first and second blades 4, 18 may include a cutting portion 32, as shown in FIGS. 16 and 17. The cutting portion 32 is preferably situated adjacent and proximal to the non-cutting, clamping portion 12, 26 and proximal to the tip 10, 24. Each cutting portion 32 has a sharpened edge 34. The cutting portions 32 of the first and second blades 4, 18 are in alignment with each other, with the sharpened edges 34 of each blade facing each other to excise tissue therebetween. Alternatively, the cutting portion can be situated adjacent to the non-cutting clamping portion 12, 26 at the end of the blades in the most distal portion of the instrument.

Preferably, each of the first and second blades 4, 18 includes a tapered edge 36. The tapered edge 36 of the first blade 4 faces in an opposite direction to that of the tapered edge 36 of the second blade 18. The tapered edges 36 of the blades are provided to perform blunt dissection of tissue at the surgical site.

The non-cutting, clamping portion 12, 26 of at least one of the first and second blades 4, 18 may include a plurality of projections 38 extending from the clamping face 30 thereof for engaging and selectively securing a surgical implant device, such as a surgical mesh 14, between the clamping faces 30 of the first and second blades 4, 18. Preferably, the projections 38 are spaced apart from one another a predetermined distance which corresponds to the distance between the interstices of the mesh 14 so that the projections 38 are received by the mesh interstices to help hold the mesh in place during the implantation procedure. Alternatively, the non-cutting, clamping portion 12 of one of the blades, such as the first blade 4, includes a plurality of projections 38 extending from the clamping face 30 thereof, and wherein the non-cutting, clamping portion 26 of the other blade, such as the second blade 18, includes a plurality of recesses 40 formed in the clamping face 30 thereof. The projections 38 of the first blade 4 are situated in alignment with respective recesses 40 of the second blade 18 when the first and second blades 4, 18 are pivotally disposed relative to each other in the first position, i.e., the clamping position. The projections 38 are provided for engaging and selectively securing a surgical implant device between the clamping faces 30 of the first and second blades 4, 18, and the corresponding recesses 40 aid in this clamping function.

In yet another alternative embodiment of the present invention, projections 38 may be formed on the clamping faces 30 of each of the first and second blades 4, 18 and, even further preferably, the projections 38 on one clamping face 30 of one blade 4 are offset from the positions of the projections 38 on the clamping face 30 of the other blade 18 so that the projections 38 extend alternately between one another. This, again, helps secure the surgical implant device between the clamping faces 30 of the two blades.

Furthermore, the tip 10, 24 of either the first or second blade 4, 18, or both blades, may include a pad 42 situated thereon which faces the tip of the other blade to increase the holding force applied by the non-cutting, clamping portions 12, 26 of the first and second blade 4, 18 on a surgical implant device held thereby.

Another feature of the surgical instrument 2 of the present invention is the provision to apply additional pressure between the inside surfaces of the blades. This is accomplished by providing a distance between the two handle sections when the device is completely closed and the surfaces of the blades are in contact with the implant, with the implant held between them.

Another feature of the surgical instrument 2 of the present invention is the provision of markings to determine the dimensions of a tissue dissection performed by the surgical instrument. More specifically, at least one of the first and second blades 4, 18, or again both blades, may include markings 44 situated along a portion of the length thereof, and preferably along the first section 6, 20 thereof. The markings 44 may include a measurement scale having numbers so that the surgeon may quickly and easily measure the depth and extent of a channel or tunnel formed in the dissected tissue, thereby avoiding the need to use a separate ruler, as is conventionally done.

The surgical instrument 2 of the present invention may be formed from a number of materials, including from a group of polymers generally used for medical devices, such as polycarbonate, Delrin™, Nylon™, polypropylene, polysolfoam, or combinations and blends of these and other similar compounds. Additionally, the surgical instrument 2 of the present invention may be made from various metals, such as stainless steel, aluminum, titanium or Nitinol™ and combinations of these metals. Furthermore, the surgical instrument 2 of the present invention may be made from combinations of polymers and metals. For example, the handle portion (i.e., second sections 16, 28) of the instrument 2 may be made from a polymer while the first sections 6, 20 of the blades are made from metal. The metal first section 6, 20 of each of the first and second blades 4, 18 adds stiffness and reduces flex, especially when the blades are brought together and are required to hold the mesh 14 or other surgical implant device in place.

Alternatively, the surgical instrument 2 of the present invention may have a metal bar or rod (not shown) embedded along at least the partial length of each of the first and second blades 4, 18 to create a more durable unit and one that does not yield under stress or strain. In yet a further alternative embodiment of the present invention, the surgical instrument 2 of the present invention may be formed where the first sections 6, 20 of the blades have clamping faces 30, tips 10, 24 and cutting edges 34 reinforced with or constructed from metal, with the rest of each blade being formed from a polymer. Such a configuration would aid in producing a thin edge for each blade which would dissect tissue easier.

The surgical instrument 2 of the present invention performs a number of individual functions, thereby eliminating the need for the surgeon to use multiple instruments. First, the surgical instrument may perform a dissection of the tissue at the surgical site.

There are two dissection techniques which this device can accomplish. The first is blunt penetration dissection. This technique is accomplished by pushing the flattened tip 10, 24 of each blade 4, 18 of the surgical instrument through certain types of tissue. The blade tips 10, 24 are not sharp like a knife would be, and so no cutting occurs. Rather, the first and second blades 4, 18 have a tapered, flattened thin tip, which ends in a dull edge. The edge separates tissue layers as it is directly advanced forward into the tissue.

The second dissection which the surgical instrument of the present invention performs is a blunt spreading dissection. For this type of dissection, the outer edges 36 of the first and second blades 4, 18 of the surgical instrument are, again, tapered to a fine but dull, non-cutting edge. These edges 36 are comparable to those of the outer edges of a scissor. Once the surgical instrument 2 has been inserted tip first into the tissue, and separates some of the tissue layers, the two blades 4, 18 are spread apart by opening the handle. As the first and second blades 4, 18 are spread, they separate more tissue layers. Preferably, the first sections 6, 20 of each of the first and second blades 4, 18 are curved similar to a Metzenbaum scissor. This aids in placement of the surgical instrument and the surgeon's familiarity with the instrument, since the Metzenbaum scissor is used extensively in many surgical procedures.

Another function performed by the surgical instrument 2 of the present invention is grasping and manipulation of the surgical implant device, such as a surgical mesh 14 used in performing a pelvic floor prolapse repair, for example. As described previously, the clamping faces 30 of the first and second blades 4, 18 are twisted approximately 25 degrees to approximately 50 degrees around the longitudinal axis of each blade. The twist is critical in producing a blade action in which a relatively small separating movement of the handles (i.e., the second sections 16, 28 of the blades) of the surgical instrument 2 causes the clamping faces 30 of the non-cutting, clamping portion 12, 26 at the first sections 6, 20 of the blades to separate completely along their length substantially instantly. Any tissue, or a surgical implant device 14, which is being held between the blades, is completely released substantially all at one time.

The advantages of such a mechanism are twofold. First, the configuration of the surgical instrument 2 of the present invention provides for a positive opening of the first section 6, 20 of the blades 4, 18 and, even more particularly, a positive separation of the tips 10, 24 and non-cutting, clamping portions 12, 26 of the blades, so that if the surgical instrument is within a narrow tissue channel, it can easily overcome any tissue pressure against it. Secondly, the channel formed of dissected tissue can be of minimal diameter and the blades 4, 18 can still open enough to release the surgical implant device in the channel.

Advantageously, the structure of the surgical instrument 2 of the present invention allows the surgical implant device 14 to be held between the non-cutting, clamping portions 12, 26 of the first and second blades 4, 18 with a high positive force, keeping it held securely. To achieve additional holding force, the second sections 16, 28 of the first and second blades 4, 18 which define the handle are kept slightly separated, as shown in FIG. 1, when the blades are initially in their clamping position. There is a slight space between the two second sections 16, 28 of the blades forming the handle. Further squeezing together hard on the two second sections 16, 28 defining the handle of the surgical instrument subsequently increases the force applied by the clamping faces 30 on the surgical implant device 14.

The surgical instrument 2 of the present invention further performs a measurement function. To correctly size the surgical implant device, such as a surgical mesh 14, the tissue cavity created from the dissection must be measured. As mentioned previously, the surgical instrument 2 of the present invention has imprinted or engraved on it graduated measurement indicator 44, such as a measurement scale with numbers. The graduated measurement indicators 44 may consist of lines arranged along at least a portion of the length of at least the first section 6, 20 of each blade 4, 18, where the indicators 44 are set at equal and specific distances from each other. The measurement scale, if optionally provided, would include numbers that correspond to distances from the distal tip 10, 24 of each of the first and second blades 4, 18. The surgical instrument 2 is either laid across a tissue dissection, or placed into a tissue channel, and the depth, length or distance of the channel can be readily determined. These distances are then compared to similar distance indicators on the implant device or implant holder or with another measurement device.

The surgical instrument 2 of the present invention also performs a cutting function. A shearing mechanism, such as described previously with respect to the cutting portion 32 provided on each of the first and second blades 4, 18, may be incorporated so that, as the second sections 16, 28 of the blades defining the handle are brought together and the first sections 6, 20 pass each other, the cutting portion 32 may contact and shear or excise any tissue or material between them. The cutting portion 32 of the blades may be positioned either between the pivot point and the non-cutting, clamping portion 12, 26 on the first section 6, 20 of each blade, or between the non-cutting, clamping portion 12, 26 and the tip 10, 24 of each blade, or, alternatively, in both such places.

Thus, the surgical instrument 2 of the present invention performs the function of dissection, measurement, and grasping, manipulating, cutting and inserting a surgical implant device, all in one instrument. The overall general shape of the surgical instrument of the present invention is similar to other instruments that the surgeon is familiar with so that the learning time for accomplishing the surgical procedures with the instrument of the present invention will be reduced. In addition, the level of confidence of the surgeon to accomplish the tasks performed by the surgical instrument 2 of the present invention is increased since he or she already knows how to manipulate instruments with similar overall physical characteristics.

The surgeon will spend less time looking for the right surgical instrument or in changing over to a different instrument to perform the various surgical procedures at hand. This will also provide a benefit to the patient, in that the surgical procedure will take less time and, accordingly, the patient's exposure time to anesthesia will be reduced.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. A surgical instrument for dissecting tissue and inserting a surgical implant device, which comprises:
a first blade having an elongated length including a first section and a second section disposed opposite the first section, the first section of the first blade including a free end having a non-cutting, clamping portion for selectively grasping a surgical implant, the second section of the first blade including a finger ring for grasping and manipulation by a surgeon;
a second blade having an elongated length including a first section and a second section disposed opposite the first section, the second section of the second blade including a free end having a non-cutting, clamping portion for selectively grasping a surgical implant, the second section of the second blade including a finger ring for grasping and manipulation by a surgeon, the first blade and the second blade being pivotally joined together at the junctures of the first and second sections of each blade to allow the first and second blades to pivotally move with respect to one another within a plane of pivotal movement, the non-cutting, clamping portion of each of the first and second blades having a clamping face, the clamping face of the first blade and the clamping face of the second blade being arranged to face each other and to selectively engage and hold therebetween a surgical implant device, the clamping faces of the first and second blades residing in parallel curved or non-curved planes and being angled to the plane of pivotal movement of the blades, the first and second blades being relatively pivotable between at least a first position, wherein the clamping faces of the first and second blades are spaced from each other a first predetermined distance to grasp and hold securely therebetween a surgical implant device, and a second position, wherein the clamping faces of the first and second blades are spaced from each other a second predetermined distance to release a surgical implant device situated therebetween, the first predetermined distance being less than the second predetermined distance.

2. A surgical instrument for dissecting tissue and inserting a surgical implant device as defined by claim 1, wherein the planes in which each of the clamping faces of the first and second blades respectively reside are disposed at about a 40 degrees angle to the plane of pivotal movement of the first and second blades.

3. A surgical instrument for dissecting tissue and inserting a surgical implant device as defined by claim 2, wherein the non-cutting, clamping portions of the first and second blades are curved.

4. A surgical instrument for dissecting tissue and inserting a surgical implant device as defined by claim 1, wherein the first section of each of the first and second blades includes a cutting portion situated adjacent to the non-cutting, clamping portion, each cutting portion having a sharpened edge, the cutting portions of the first and second blades being in alignment with each other and with the sharpened edges of each blade facing each other to excise tissue therebetween.

5. A surgical instrument for dissecting tissue and inserting a surgical implant device as defined by claim 1, wherein each of the first and second blades includes a flattened tip at the free end thereof to perform blunt dissection of tissue.

6. A surgical instrument for dissecting tissue and inserting a surgical implant device as defined by claim 1, wherein each of the first and second blades includes a tapered edge, the tapered edge of the first blade facing in an opposite direction to that of the tapered edge of the second blade, the tapered edges of the blades being tapered to perform blunt dissection of tissue.

7. A surgical instrument for dissecting issue and inserting a surgical implant device as defined by claim 1, wherein the first section of at least one of the first and second blades includes markings situated thereon to sense the dimensions of a tissue dissection performed by the surgical instrument.

8. A surgical instrument for dissecting tissue and inserting a surgical implant device as defined by claim 7, wherein the markings include a measurement scale having numbers.

9. A surgical instrument for dissecting tissue and inserting a surgical implant device as defined by claim 1, wherein the non-cutting, clamping portion of at least one of the first and second blades includes a plurality of projections extending from the clamping face thereof for engaging and selectively securing a surgical implant device between the clamping faces of the first and second blades.

10. A surgical instrument for dissecting tissue and inserting a surgical implant device as defined by claim 1, wherein the non-cutting, clamping portion of the first blade includes a plurality of projections extending from the clamping face thereof, and wherein the non-cutting, clamping portion of the second blade includes a plurality of recesses formed in the clamping face thereof, the projections being in alignment with respective recesses when the first and second blades are pivotally disposed relative to each other in the first position, the projections being provided for engaging and selectively securing a surgical implant device between the clamping faces of the first and second blades.

11. A surgical instrument for dissecting tissue and inserting a surgical implant device as defined by claim 1, wherein each of the non-cutting, clamping portions of the first and second blades includes a plurality of projections extending from the clamping face thereof for engaging and selectively securing a surgical implant device between the clamping faces of the first and second blades.

12. A surgical instrument for dissecting tissue and inserting a surgical implant device as defined by claim 11, wherein the projections of the first blade are offset from the position of the projections of the second blade.

13. A surgical instrument for dissecting tissue and inserting a surgical implant device as defined by claim 1, wherein each of the first and second blade includes a tip situated at the free end thereof, and wherein the tip of at least the first blade includes a pad situated thereon and facing the tip of the second blade to increase the holding force applied by the non-cutting, clamping portions of the first and second blades to a surgical implant device held thereby.

14. A surgical instrument for dissecting tissue and inserting a surgical implant device, which comprises:
a first blade having an elongated length comprised of a first section and a second section disposed opposite the first section, the first section of the first blade including a free end having a flattened tip for blunt dissection of tissue and a non-cutting, clamping portion situated adjacent to and encompassing the flattened tip for selectively grasping a surgical implant device, the second section of the first blade including a finger ring for grasping and manipulation by a surgeon;
a second blade having an elongated length including a first section and a second section disposed opposite the first section, the first section of the second blade including a free end having a flattened tip for blunt dissection of tissue and a non-cutting, clamping portion situated adjacent to and encompassing the flattened tip for selectively grasping a surgical implant device, the second section of the second blade including a finger ring for grasping and manipulation by a surgeon, the first blade and the second blade being pivotally joined together at the junctures of the first and second sections of each blade to allow the first and second blades to pivotally move with respect to one another within a plane of pivotal movement, the non-cutting, clamping portion of each of the first and second blades having a clamping face, the clamping face of the first blade and the clamping face of the second blade being arranged to face each other and to selectively engage and hold therebetween a surgical implant device, the clamping faces of the first and second blades residing in parallel curved or non-curved planes and being angled to the plane of pivotal movement of the blades, the first and second blades being relatively pivotable between at least a first position, wherein the clamping faces of the first and second blades are spaced from each other a first predetermined distance to grasp and hold securely therebetween a surgical implant device, and a second position, wherein the clamping faces of the first and second blades are spaced from each other a second predetermined distance to release a surgical implant device situated therebetween, the first predetermined distance being less than the second predetermined distance, the planes in which each of the clamping faces of the first and second blades respectively reside being disposed at about a 40 degrees angle to the plane of pivotal movement of the first and second blades, each of the first and second blades including a tapered edge, the tapered edge of the first blade facing in an opposite direction to that of the tapered edge of the second blade, the tapered edges of the blades being tapered to perform blunt dissection of tissue, the first section of at least one of the first and second blades including markings situated thereon to sense the dimensions of a tissue dissection performed by the surgical instrument.
